(19) United States
(12) Patent Application Publication
(10) Pub. No.: US 2004/0131595 A1
(43) Pub. Date: Jul. 8, 2004

(54) Treating carcinoid neoplasms with therapeutic viruses

(75) Inventors: Robert M. Lorence, Bethesda, MD (US); Pierre Major, Hamilton (CA)

Correspondence Address: LEWIS J. KREISLER, LEGAL DEPARTMENT, 930 CLOPPER ROAD, GAITHERSBURG, MD, 20878, US

(73) Assignee: Wellstat Biologics Corporation

(21) Appl. No.: 10/700,143

(22) Filed: Nov. 3, 2003

Related U.S. Application Data
(60) Provisional application No. 60/423,952, filed on Nov. 5, 2002.
(60) Provisional application No. 60/457,034, filed on Mar. 24, 2003.

(51) Int. Cl. A61K 48/00

(52) U.S. Cl. 424/93.2; 424/93.6

(57) ABSTRACT

A negative-stranded RNA virus is used to treat a mammalian subject having a carcinoid tumor.

This application claims the benefit of U.S. Provisional Application No. 60/423,952, filed Nov. 5, 2002 and 60/457,034 filed Mar. 24, 2003, the content of which is incorporated herein by reference.

### BACKGROUND OF THE INVENTION

The treatment of neoplasms, including neuroendocrine carcinomas with viruses is disclosed in WO 00/62735. Carcinoid tumors are a type of neuroendocrine tumor. (See WO 00/62735, page 32.) The administration of a desensitizing dose of an oncolytic virus before higher subsequent doses is disclosed in WO 00/62735 (pages 35-36). See also Pecora, et al., J. Clin. Oncol. (May 2002) 20(9):2251-2266; and Bergsland, et al., J. Clin. Oncol. (May 2002) 20(9): 2220-2222.

The administration of oncolytic viruses using an intravenous pump, syringe pump, intravenous drip or slow injection over the course of 4 minutes to 24 hours, for example over the course of 20 to 60 minutes, is disclosed in WO 00/62735 (page 36, lines 16-19).

### SUMMARY OF THE INVENTION

This invention provides a method for treating a mammalian subject having a carcinoid tumor, comprising administering to the subject an amount of a therapeutic virus effective to treat the condition, wherein the virus is a negative-stranded RNA virus.

This invention is based on the finding that a negative-stranded RNA virus, such as Newcastle Disease Virus, is effective to lessen the symptoms of carcinoid syndrome and to decrease carcinoid tumor mass in a patient.

### DETAILED DESCRIPTION OF THE INVENTION

As used herein the transitional term “comprising” is open-ended. A claim utilizing this term can contain elements in addition to those recited in such claim. Thus, for example, the claims can read on treatment regimens that also include other therapeutic agents or therapeutic virus doses not specifically recited therein, as long as the recited elements or their equivalent are present.

As used herein “NDV” is an abbreviation for Newcastle Disease Virus. As used herein “DLT” is an abbreviation for dose limiting toxicity. As used herein the term “plaque-forming unit” (PFU) means one infectious virus particle. As used herein “BPFU” means billion PFUs. As used herein “PP” means plaque-purified. Thus, for example PPMK107 means plaque-purified Newcastle Disease virus strain MK107. As used herein “PFU/m²”, which is a standard unit for expressing dosages, means PFUs per square meter of patient surface area. As used herein the term “replication-competent” virus refers to a virus that produces infectious progeny in cancer cells.

“Carcinoid syndrome” is a diagnosis that can be made when a patient with proven carcinoid tumor also presents certain other symptoms, especially one or more of diarrhea, flushing and fatigue. Carcinoid syndrome is found in a subset of carcinoid tumor patients.

In accordance with the methods of this invention the therapeutic Newcastle disease virus utilized can be of low (lentogenic), moderate (mesogenic) or high (velogenic) virulence. The level of virulence is determined in accordance with the Mean Death Time in Eggs (MDT) test. (Alexander, “Chapter 27: Newcastle Disease” in Laboratory Manual for the Isolation and Identification of Avian Pathogens, 3^{rd} ed., Purchase, et al. eds. (Kendall/Hunt, Iowa), page 117.) Viruses are classified by the MDT test as lentogenic (MDT>90 hours); mesogenic (MDT from 60-90 hours); and velogenic (MDT<60 hours).

Any conventional negative-stranded RNA virus can be utilized in accordance with this invention to treat a mammalian subject having a carcinoid tumor. In an embodiment of the method of this invention, the virus is a replication-competent oncolytic virus. In progressively more specific embodiments, the replication-competent oncolytic virus is a Paramyxovirus, for example a Newcastle Disease Virus, and more specifically a mesogenic strain of Newcastle Disease Virus.

In accordance with this invention, any conventional route or technique for administering viruses to a subject can be utilized. In one embodiment of this invention, the virus is administered systemically, for example intravenously. For intravenous administration of a therapeutic virus in accordance with this invention, preferably the virus is a mesogenic strain of Newcastle Disease Virus.

It has been found that undesired side effects can be decreased by controlling the rate at which the virus is administered. When administering a mesogenic strain of Newcastle Disease Virus by the intravenous route, is preferable for a dose of the virus to be administered over an administration time period of up to 24 hours; and the dose to be administered at a rate of up to 7.0×10⁸ PFU per square meter of patient surface area in any ten minute sampling time period within the administration time period. More preferably, the rate at which the dose is administered is up to 2.0×10⁸ PFU per square meter of patient surface area in any ten minute sampling time period within the administration time period. Generally it is convenient to select the rate of administration so that the administration time period is at least 1 hour. Still fewer side effects are generally observed when the administration time period is at least 3 hours.

In one embodiment of this invention, the therapeutic virus is administered to the subject in one or more cycles, wherein at least one cycle comprises administering sequentially one or more desensitization doses of the virus followed by administering one or more escalated doses of the virus, wherein the amount of the virus in each escalated dose is higher than the amount of virus in each desensitization dose. In a more specific embodiment, the cycle comprises one desensitization dose of from 1.2×1010 PFU to 4.8×10¹⁰ PFU per square meter of patient surface area, and one or more escalated doses of from 2.4×10¹⁰ PFU to 1.2×10¹¹ PFU per square meter of patient surface area. In a more specific embodiment the desensitization dose is about 2.4×10 PFU per square meter of patient surface area, and the one or more escalated doses are from 4.8×10¹⁰ to 1.2×10¹¹ PFU per square meter of patient surface area. In a still more specific embodiment the desensitization dose is about 2.4×10¹⁰ PFU per square meter of patient surface area, and the one or more escalated doses are about 4.8×10¹⁰ PFU per square meter of patient surface area. A regimen utilizing desensitization and escalated doses can be combined with the technique described above of controlling the rate of administration of one or more of the doses. It is especially helpful to control the rate at which the first desensitization dose of the virus is administered.

The subject that is treated in accordance with this invention can be either a human subject or a non-human mammalian subject.

Although monitoring the treatment is not an essential aspect of the invention, there are techniques for measuring the therapeutic effects of the treatment. These include, measuring the size of the tumor after administration of the virus, and a decrease in tumor size is a positive result. Alternatively the level of 5-hydroxyindole acetic acid (5HIAA) in urine of the subject is measured after administration of the virus, and a decrease in the level of 5HIAA is a positive result. In cases where the subject had carcinoid syndrome prior to administration of the therapeutic virus, successful treatment can be monitored by a decrease in one or more symptoms (e.g. diarrhea, flushing, fatigue) of carcinoid syndrome after administration of the virus.

The invention will be better understood by reference to the following examples, which illustrate but do not limit the invention described herein. In the following examples the NDV used was a triple-plaque purified attenuated (mesogenic) version of the MK107 strain of Newcastle Disease Virus, described more fully in International Patent Publication WO 00/62735, published Oct. 26, 2000 (Pro-Virus, Inc.). The entire content of WO 00/62735 is hereby incorporated herein by reference.

Two of the symptoms of carcinoid syndrome, diarrhea and/or flushing, are commonly controlled with octreotide. Thus, a decrease in the dose of octreotide needed to control such symptoms provides a quantifiable way of measuring a decrease in carcinoid syndrome symptoms in those patients experiencing diarrhea and/or flushing. Preferably the octreotide can be discontinued and the diarrhea/flushing is controlled without octreotide. See Example 2, Patient 2102.

### EXAMPLES

### Example 1

A 63 year old woman with advanced carcinoid metatastatic to the liver and mesentery had had prior treatment with 5-fluorouracil with disease progression noted in 2001 in spite of etoposide therapy. In month 1, she was having increasing symptoms of carcinoid syndrome consisting of significant fatigue, diarrhea, and tearing in spite of increasing doses of octeotide (Sandostatin™ LAR 20 mg) administered subcutaneously on a monthly basis.

In month 3, she started cycles of NDV therapy consisting of six doses administered over 2 weeks followed by one week off therapy. The first dose of 12 billion PFU per meter squared was administered over 3 hours intravenously. In each cycle, her next 5 doses were at 24 billion PFU per meter squared given over 1 hour intravenously.

In response to NDV therapy, she has had marked symptomatic improvement in her carcinoid syndrome consisting of lessening in her bouts of diarrhea and lessening in her fatigue. Her urine tumor marker (5-hydroxyindole acetic acid, 5HIAA) was measured in month 5, and it had decreased 43% from her level taken in month 1. A 6.5 cm mesenteric mass was noted by ultrasound exam to have decreased by >90% following NDV treatment (from 6.4×3.0×3.0 cm to 2.0×0.8×0.8 cm).

### Example 2

Methods

Schedule of Dosing and Dose Amounts:

Courses 1-6

For first 2 courses, six doses were given to cancer patients over a 2-week time period followed by one week without NDV treatment for a 21-day cycle.

Schedule:

| | | |
|---|---|---|
| Dose 1 | Day 0 | Administered over 3 hours by intravenous infusion |
| Dose 2 | Day 3 | Administered over 1 hour by intravenous infusion |
| Dose 3 | Day 7 | Administered over 1 hour by intravenous infusion |
| Dose 4 | Day 9 | Administered over 1 hour by intravenous infusion |
| Dose 5 | Day 11 | Administered over 1 hour by intravenous infusion |
| Dose 6 | Day 14 | Administered over 1 hour by intravenous infusion |

For the next 4 courses, six doses were given to cancer patients over a 2 week time period followed by one week without NDV treatment for a 21 day cycle.

Schedule:

| | | |
|---|---|---|
| Dose 1 | Day 0 | Administered over 1 hour by intravenous infusion |
| Dose 2 | Day 2 | Administered over 1 hour by intravenous infusion |
| Dose 3 | Day 4 | Administered over 1 hour by intravenous infusion |
| Dose 4 | Day 7 | Administered over 1 hour by intravenous infusion |
| Dose 5 | Day 9 | Administered over 1 hour by intravenous infusion |
| Dose 6 | Day 11 | Administered over 1 hour by intravenous infusion |

Courses 7+

Beginging with 7^{th} course of NDV, subsequent courses consisted of only 3 doses given in one week followed by 3 weeks without receiving NDV before beginning the next course for a 4-week cycle.

| | | |
|---|---|---|
| Dose 1 | Day 0 | Administered over 1 hour by intravenous infusion |
| Dose 2 | Day 2 | Administered over 1 hour by intravenous infusion |
| Dose 3 | Day 4 | Administered over 1 hour by intravenous infusion |

Dose Amounts By Cohort:

| | | | Doses 2-6 [for |
|---|---|---|---|
| | | | courses 1-6] |
| | | Dose 1 | OR Doses 2-3 |
| Cohort | # | (billion | [for courses 7 |
| # | of Patients | PFU/m²) | or greater] |
| 1 | 3 | 12 | 24 |
| 2 | 6 | 24 | 48 |
| 3 | 3 | 24 | 96 |
| 4 | 4 | 24 | 120 |
| | treated to date | | |
| | (up to 6 will be | | |
| | enrolled) | | |

In this experiment Dose 1 was not escalated higher than 24 billion PFU/m² because of asymptomatic hypotension and moderate fever observed at this dose in patients of Cohort 2. However an initial dose as high as 48 billion PFU/m² can be given in a hospital setting because a hospital can provide adequate management of the anticipated symptomatic hypotension and fever with such initial dose.

Prior to First Dose of First Course:

Acetaminophen (650 mg) was given immediately prior to dosing. Beginning with patient 2304 (last patient of cohort #3), an additional ibuprofen dose (400 mg) was given immediately prior to dose 1 for further prophylactic control of fever.

Beginning with patient 2201 (first patient of cohort #2), an intravenous dose of ondansetron (8 mg) was also given immediately prior to dosing for prophylactic control of nausea.

After their First Dose of First Course:

Acetaminophen (650 mg) was given 4, 8, and 12 hours after dosing. Ibuprofen (400 mg) was given 6, 12, 18, 24 hours after dosing. Ondansetron (8 mg) was given 12 and 24 hours after dosing. Patients were kept in the hospital overnight for monitoring and given IV fluids at 200 cc/h for 24 hours, starting when the pre-medications were given. For the day after discharge, they were given another liter of IV fluids at home.

Prior to Second Dose of First Course:

Acetaminophen (650 mg) was given immediately prior to dosing. Beginning with patient 2201, IV Dolasetron (100 mg) was given immediately prior to dosing. Beginning with patient 2304, Ibuprofen (400 mg) was also given immediately prior to dosing.

After Second Dose of First Course:

Acetaminophen (650 mg) was given 4, 8, and 12 hours after dosing. Ibuprofen (400 mg) was given 6, 12, 18, 24 hours after dosing. Patients were given a 500 ml to 1 liter of IV fluids with dosing. For each of the next 3 days, they were given another liter of IV fluids at home.

Prior to Each Successive Dose of First Course:

Acetaminophen (650 mg) was given immediately prior to dosing. Beginning with patient 2304, Ibuprofen (400 mg) was also given immediately prior to dosing.

After Each Successive Dose of First Course:

Acetaminophen (650 mg) was given 4, 8, and 12 hours after dosing. Ibuprofen (400 mg) was given 6, 12, 18, 24 hours after dosing.

Description of Patients

Cohort 1 (12/24×5)

Patient 2101 (59 year old woman with colon cancer); Stable Disease for 6+months

First dose given: 7-8-02

# of Courses received: 8

Description: After NDV treatment, this patient has had increased cystic fluid in the pelvis associated with her cystic pelvic tumor mass. All of the other mets showed tumor reduction (but not enough to call a response). The fluid associated with the pelvic mass was aspirated revealing no evidence of malignant cells, only necrotic material and inflammatory cells. The patient had stable disease for 6 months (after receiving a total of 8 courses of NDV) and then had evidence of tumor progression (as evidenced by continued growth of the cystic pelvic mass and hydronephrosis).

Patient 2102 (63 year old woman with malignant carcinoid. Note: same patient as Example 1); Minor Radiographic Response; Major Biochemical Response Ongoing; Now on-study for 8+months

First dose given: 7-22-02

# of Courses received: 10+

Description: This patient had carcinoid syndrome (mainly diarrhea and fatigue with some flushing) and was on octreotide before starting the study with incomplete control of the diarrhea. After starting NDV treatment she was noted to have:

1) Complete symptomatic improvement. She was taken off octreotide and remained off for 100 days with no signs of diarrhea/flushing. Her long-acting injection at return of symptoms at 100 days resulted in complete freedom form symptoms for a further 114 days.

2) A drop in 5HIAA of 43% comparing pre-NDV treatment levels with octreotide to post-NDV treatment levels while off octreotide.

3) A>90% reduction in her mesenteric mass (overall minor response based on minimal changes in the size of her liver mets)

She is still on study.

Patient 2103 (40 year old woman with borderline ovarian carcinoma with peritoneal mets); Stable Disease for 4 months; then tumor progression (3 new tumor nodules)

First dose given: 7-29-02

# of Courses received: 6

Description: After first course, the CA-125 showed a 50% decline.

Cohort 2 (24/48×5)

Patient 2201 (61 year old man with rectal cancer); Partial response confirmed and ongoing; on study now for 7+months

First dose given: 8-19-02

# of Courses received: 7+

Description: After the first CT scan, a 50% tumor reduction was noted. The PR was confirmed on the second scan. The third scan showed a 75% overall reduction in tumor size from baseline. His CEA also showed a 70% reduction initially.

Patient 2202 (35 year old man with rectal cancer and pelvic mets); 50% reduction in tumor size (Partial response), still on study

First dose given: 9-9-02

# of Courses received: 5

Description: This patient developed sepsis after dose 1 of course 1. The blood cultures showed viridans strep (Strep salivarius). This patient prior to dose 1 had had a vigorous teeth cleaning which is a possible source for sepsis from this oral bacteria. The rest of his doses during course 1 were held. After responding to antibiotic therapy, the patient restarted NDV treatment. During course 2, he developed worsening sciatic pain and was started on high dose high frequency Dilaudid (hydromorphone) and did not take adequate stool softeners. He subsequently developed small bowel obstruction along with subsequent infection by E. coli, believed likely due to non-aseptic care of his ileostomy. The patient has recently developed a rectal fistula, underwent surgical repair. Follow up scans after 5 cycles and prior to surgery showed a 50% reduction in overall tumor size (partial response) and the patient continues on study.

Patient 2203—enrolled but never dosed

Patient 2204 (50 year old man with colon cancer); stable for 2 months then developed tumor progression

First dose given: 10-7-02

# of Courses received: 3

Patient 2205 (45 year old man with carcinoid of the larynx): Minor response ongoing; on study now for 5+months

First dose given: 10-21-02

# of Courses received: 6+

Description: This patient's laryngeal tumor decreased 30% from baseline after 2 cycles. Currently awaiting evaluation after 6 cycles.

Patient 2206 (56 year old woman with in-transit metastatic melanoma): Partial Response ongoing; on study now for 4+months

First dose given: 10-28-02

# of Courses received: 6+

Description: This patient had >30 in-transit skin mets, the 10 largest of which have been tracked for size. These show a ˜67% decrease in the sum of the tumor areas with some lesions completely regressed. Interestingly, the patient notes that the day after dosing lesions get inflamed (red) and this resolves by the next day. The patient currently feels well.

Patient 2207 (58 year old man with colon cancer): Recently completed 2 cycles; still on study.

First dose given: 11-4-02

# of Courses received: 6

Description: He completed 6 cycles of NDV without incident and had stable disease on his first scan.

Cohort 3 (24/96×5)

Patient 2301 (67 year old woman with ovarian cancer): Tumor progressed after 2 cycles and patient taken off study.

First dose given: 11-18-02

# of Courses received: 2

Patient 2302 enrolled but never treated

Patient 2303 (45 year old woman with ovarian cancer): On study for 3 months with stable disease.

First dose given: 12-2-02

# of Courses received: 4+

Description: During her 3^{rd} high dose of 96 billion PFU/m2, she experienced severe chest pain with rigors and rigors-associated hypoxia (Jan. 3, 2003, dose 4 of cycle 2). The pain resolved when the infusion was ended. She was also treated with Demerol, nitrospray and oxygen. For her next several doses, she was given prophylactic Benedryl, the infusion time was increased to 2 hours and she subsequently had no recurrence of this infusion-related side effects. She currently needs no pre-treatment Benedryl and the infusion time is 1 hour. She has had stable disease now for 3+ months.

Patient 2304 (45 year old man with round cell sarcoma of the right thigh and pelvic bone mets). Recently completed 2 courses. Evaluation pending. He required admission for pain control related to his bone mets.

First dose given: 1-20-03

# of Courses received: 2

Cohort 4 (24/120×5)

Patient 2401 (62 year old man with cancer of the GE (gastro-esophageal) junction with liver mets). Recently completed 2 courses. He has pain in the liver where metastases are located. He also has had vomiting and decreased appetite. He has required intermittent on-going home hydration for prevention of dehydration. Evaluation pending.

First dose given: 2-03-03

# of Courses received: 2

Patient 2402 (33 year old woman with recurrent cervical cancer). Recently completed the first course and tolerated treatment well. Mild fatigue and nausea were only symptoms.

First dose given: 2-17-03

# of courses given: 1+

Patient 2403 Patient enrolled but not treated.

Patient 2404 (52 year old man with colon cancer and liver metastases). This patient has recently completed the 1^{st} course of NDV treatment and tolerated treatments well with only moderate fatigue and some emesis.

First dose given: 2-24-03

# of courses given: 1

Patient 2405 (53 year old woman with colon cancer and liver metastases). This patient recently started her first course. Moderate fatigue was noted. She experienced a mild infusion reaction during the 3rd dose that resolved with Benadryl and a longer infusion time. No Benadryl was given with 5^{th} dose but the longer infusion time was maintained.

First dose given: 3-03-03

# of courses given: 1

What is claimed is:

1. A method for treating a mammalian subject having a carcinoid tumor, comprising administering to the subject an amount of a therapeutic virus effective to treat the condition, wherein the virus is a negative-stranded RNA virus.

2. The method of claim 1, wherein the virus is a replication-competent oncolytic virus.

3. The method of claim 2, wherein the oncolytic virus is a Paramyxovirus.

4. The method of claim 3, wherein the Paramyxovirus is a Newcastle Disease Virus.

5. The method of claim 4, wherein the virus is a mesogenic strain of Newcastle Disease Virus.

6. The method of claim 1, wherein the virus is administered systemically.

7. The method of claim 6, wherein the virus is administered intravenously.

8. The method of claim 7, wherein the virus administered is a mesogenic strain of Newcastle Disease Virus.

9. The method of claim 8, wherein the virus is administered over an administration time period of up to 24 hours; and the dose is administered at a rate of up to 7.0×10⁸ PFU per square meter of patient surface area in any ten minute sampling time period within the administration time period.

10. The method of claim 9, wherein the rate is up to 2.0×10⁸ PFU per square meter of patient surface area in any ten minute sampling time period within the administration time period.

11. The method of claim 9, wherein the administration time period is at least 1 hour.

12. The method of claim 11, wherein the administration time period is at least 3 hours.

13. The method of claim 8, wherein the therapeutic virus is administered to the subject in one or more cycles, wherein at least one cycle comprises administering sequentially one or more desensitization doses of the virus followed by administering one or more escalated doses of the virus, wherein the amount of the virus in each escalated dose is higher than the amount of virus in each desensitization dose.

14. The method of claim 13, wherein the cycle comprises one desensitization dose of from 1.2×10¹⁰ PFU to 4.8×10¹⁰ PFU per square meter of patient surface area, and one or more escalated doses of from 2.4×10¹⁰ PFU to 1.2×10¹⁰ PFU per square meter of patient surface area.

15. The method of claim 14, wherein the desensitization dose is about 2.4×10¹⁰ PFU per square meter of patient surface area, and the one or more escalated doses are about 4.8×10¹⁰ PFU per square meter of patient surface area.

16. The method of claim 1, wherein the subject is a human subject.

17. The method of claim 1, wherein the subject is a non-human mammal.

18. The method of claim 1, wherein the size of the tumor decreases after administration of the virus.

19. The method of claim 1, wherein the level of 5-hydroxyindole acetic acid in urine of the subject decreases after administration of the virus.

20. The method of claim 1, wherein the subject has carcinoid syndrome and one or more symptoms of carcinoid syndrome are decreased after administration of the virus.

21. The method of claim 20, wherein the one or more symptoms of carcinoid syndrome are selected from the group consisting of diarrhea, flushing, and fatigue.

22. The method of claim 20, wherein the symptom of carcinoid syndrome comprises one or both of diarrhea and flushing; before beginning the therapeutic virus treatment octreotide was administered to the patient to control the symptom; and the decrease in the symptom of carcinoid syndrome is measured by a decrease in the dose of octreotide needed to control the symptom.

23. The method of claim 22, wherein the symptom is controlled without octreotide.
